(19) United States
(12) Patent Application Publication
(10) Pub. No.: US 2008/0008664 A1
(43) Pub. Date: Jan. 10, 2008

(54) METHOD OF REDUCING INFECTION

(75) Inventors: Gilbert A. Leveille, Denville, NJ (US); Steve Zibell, Tinley Park, IL (US); Kathy McMahon, Evanston, IL (US)

Correspondence Address: BELL, BOYD & LLOYD LLP, P.O. Box 1135, CHICAGO, IL, 60690, US

(73) Assignee: WM. WRIGLEY JR. COMPANY, Chicago, IL (US)

(21) Appl. No.: 11/456,411

(22) Filed: Jul. 10, 2006

(51) Int. Cl. A61K 9/68; A61K 36/61; A61K 36/53; A61K 36/537; A61K 36/328; A61K 36/534; A61K 36/28

(52) U.S. Cl. 424/48; 424/745; 424/746; 424/748; 424/764; 424/742; 424/747

(57) ABSTRACT

A method of reducing infection is provided. The method includes administering a chewing gum to a subject having an infection. The infection may include a nosocomial infection. The chewing gum may be administered to a subject lacking proper oral care.

### BACKGROUND

The present invention relates to methods of preventing an infection.

Infection is characterized by an invasion and/or multiplication of pathogenic microorganisms in a bodily part or tissue, which may produce subsequent tissue injury. An infection may progress to overt disease through a variety of cellular or toxic mechanisms.

Due to the emergence of antibiotic resistant strains of pathogens from routine use of anti-microbial agents, infections have become significant, especially in health care facilities where patients with weakened immune systems are exposed to a high concentration of other sick patients, medical staff moving from patient to patient spreading pathogens between patients, and medical procedures that bypass the body's natural protective barriers. Infections resulting from medical treatment such as treatments administered in a health care facility are commonly known as nosocomial or iatrogenic infections. In the United States alone, it has been estimated that as many as one in ten hospital patients acquires a nosocomial infection, up to 1.5 to 2 million patients a year, leading to tens of thousands of deaths each year.

The most common and most recognized way of maintaining proper oral hygiene is through regular teeth brushing and flossing. However, a patient in a health care facility may be unable to exercise proper oral hygiene for various reasons throughout most, if not all, of the duration of a stay in the facility. In the absence of proper oral care, a biofilm known as dental plaque forms on the surface of the teeth, especially along the gingival margin and interdental spaces. When plaque remains on the teeth for more than approximately seventy-two hours, the plaque hardens into tartar or calculus which cannot be completely removed by brushing and flossing. Plaque provides a surface to which the many species of bacteria in the oral cavity can adhere causing infection of gingival tissues, known as gingivitis. Within days of neglect or improper oral care, the dental plaque biofilm may further expand to populate the subgingival space, resulting in a bacterial infection known as periodontitis or periodontal disease. Since, the average stay of the nearly thirty-five million Americans currently admitted in a hospital is an average of more than five days, and, in many cases, much longer, there is sufficient time for lack of proper oral care to allow an oral infection to occur.

In addition to illness or hospitalization, other situations may prevent a subject from exercising proper oral care. Brushing and flossing teeth, for example, can often be inadequate to prevent, gingival inflammation and periodontal infection due to causes such as improper technique and portions of the dentition and gums that are inaccessible to the toothbrush. In addition, individuals may not be able or willing to brush and floss teeth regularly and/or properly for other reasons. Additional situations preventing a subject from exercising proper oral care may include lack of access to proper oral care, inconvenience, non-compliance or otherwise.

To promote oral health, attempts have been made to deliver active agents or medicaments to the oral cavity such as zinc salts, anti-microbial agents, oxidants, analogues of victamide, halogen ions, folic acid, coenzyme Q10, etc. However, efficacy, absorption, metabolism, release, bioavailability, side effects, regulation and cost associated with pharmaceuticals and medicaments delivered to the oral cavity remain problems.

### SUMMARY

The present invention relates to a method of preventing infection. In particular, the present invention relates to the administration of a chewing gum to an individual having an oral infection to reduce infection in the oral cavity of the individual in order to prevent a secondary infection in one or more areas other than the oral cavity.

To this end, in an embodiment, a method of preventing infection is provided. The method includes providing a therapeutically effective amount of a chewing gum without a medicament to a subject having an oral infection.

In an embodiment, the secondary infection includes a nosocomial or iatrogenic infection.

In an embodiment, the chewing gum is provided to a patient in a health care facility.

In an embodiment, the chewing gum is provided to a subject lacking proper oral care.

In an embodiment, the chewing gum is provided to a subject as a supplement to oral care.

In an embodiment, the chewing gum includes a flavor selected from the group consisting of an herbal flavor, a lemon flavor, a peppermint flavor and combinations thereof.

In an embodiment, the flavor is present in an amount of about 0.2% to about 5% by weight of the chewing gum.

In an embodiment, the herbal flavor includes an herbal extract selected from the group consisting of eucalyptus, clary sage, marjoram, rosemary, thyme, chamomile, lavender, myrrh and combinations thereof.

In an embodiment, the chewing gum is provided to the subject after a meal.

In another embodiment, a method of treating an oral infection is provided. The method includes providing a therapeutically effective amount of a chewing gum without an infection-reducing medicament to a subject at risk of aquiring a secondary infection in addition to the oral infection.

In an embodiment, the oral infection includes gingivitis.

In an embodiment, the secondary infection includes a nosocomial or iatrogenic infection.

In a further embodiment, a method of reducing the risk of acquiring a secondary infection is provided. The method includes providing a therapeutically effective amount of a chewing gum without an infection-reducing medicament to a subject having an oral infection.

In an embodiment, the secondary infection includes a nosocomial or iatrogenic infection.

In an additional embodiment, a method of reducing the risk of acquiring a nosocomial or iatrogenic infection is provided. The method includes providing a therapeutically effective amount of a chewing gum without a medicament to a subject in a health care facility.

It is therefore an advantage of the methods of the present invention to provide a chewing gum that is effective in preventing infection in the body by improving oral health.

Another advantage includes providing a method of preventing infection without the side effects associated with the administration of pharmacologic and therapeutic agents.

Yet another advantage includes providing a method of preventing infection in a subject without requiring the physical motions associated with brushing and flossing.

Additional features and advantages are described herein, and will be apparent from, the following Detailed Description.

### DETAILED DESCRIPTION

The present invention relates to a method of reducing the risk of infection. In particular, the present invention relates to the administration of a chewing gum to an individual in order to reduce infection and inflammation in the oral cavity of the individual which leads to infection in areas other than the oral cavity.

Inadequate or improper oral care allows a biofilm to form on the teeth of the individual. As the biofilm develops, numerous bacteria accumulate at vulnerable areas of the gum-tooth interface, and bacterial metabolites are released into the gingival epithelium. These bioactive metabolites initiate an immune response leading to production of cytokines and infiltration of the gingival epithelium with neutrophils. Neutrophils increase the permeability of the gingival blood vessels to plasma proteins which invade the surrounding tissue, producing the redness, swelling, sensitivity and bleeding associated with gingivitis. Permeable blood vessels also allow bacteria and their metabolites to reach the bloodstream. The epithelium is also stimulated to produce bioactive mediators involved in further recruitment of other immune cells, such as T-cells and monocytes, and to further produce pro-inflammatory cytokines. This sustained immune response leads to chronic gingivitis. The continued presence of dental plaque allows the bacteria to continue to grow, spread and become more pathogenic, resulting in periodontitis or periodontal disease.

The effects of sustained increased levels of oral bacteria and chronic inflammation of the surrounding gingival tissue associated with gingivitis may directly or indirectly cause systemic effects beyond the oral cavity where the condition originates. For example, bacteria from the sulcus of the periodontium may be released into the bloodstream to cause bacteremia, ingested into the gastrointestinal tract, or aspirated into the respiratory tract where infection can occur. In fact, even disturbances in the oral cavity, such as tooth brushing, flossing and chewing, may increase the likelihood of oral pathogens associated with the local infection in the periodontal tissue to spread beyond the oral cavity. In addition, locally-produced inflammatory mediators may be released into systemic circulation, and bacteria associated with gingival infection may stimulate an autoimmune response to cause a systemic pro-inflammatory response. For example, bacteria, such as Serratia marcescens, has been shown to populate subgingival biofilm and has been implicated as a cause of nosocomial infection. Consequently, there appears to be a relationship between oral infection and systematic infections in other parts of the body.

It has been surprisingly found that chewing gum actively decreases plaque, gingivitis and bleeding associated with gingivitis in the oral cavity. Therefore, methods of the present invention may include, for example, administering chewing gum to a subject having an oral infection to prevent systemic infections that can lead to a variety of diseases. In particular, the method can include, for example, administering chewing gum to a subject at risk for developing nosocomial or iatrogenic infection.

As referred to herein, a nosocomial or iatrogenic infection includes any infection which directly or indirectly results from medical treatment such as in a hospital, nursing home, hospice, assisted living facility, extended care facility, rehabilitation facility, psychiatric facility or any other long- or short-term health care facility. The infection may be secondary to the original condition of the patient.

Pathogens implicated in nosocomial infections include, but are not limited to, any commensal bacteria such as cutaneous Staphylococcus epidermidis and Escherichia coli found in the normal endogenous flora of healthy humans. Nosocomial pathogens may also include pathogenic bacteria such as any anaerobic gram-positive rods (e.g. Clostridium), gram-positive bacteria e.g. (Staphylococcus aureus and beta-hemolytic streptococci), gram-negative bacteria: Enterobacteriacae (e.g. E. coli, Proteus, Klebsiella, Enterobacter, Serratia marcescens, and Pseudomonas spp.) and any other bacteria such as Legionella. Nosocomial pathogens may further include any viruses such as hepatitis B and C viruses, respiratory syncytial virus (RSV), rotavirus, enteroviruses, cytomegalovirus (CMV), HIV, Ebola, influenza viruses, herpes simplex virus, and varicella-zoster virus. In addition, nosocomial pathogens may include parasites, such as Giardia lamblia, fungi, opportunistic parasites, such as Candida albicans, Aspergillus spp., Cryptococcus neoformans, Cryptosporidium, Aspergillus spp., and ectoparasites such as Sarcoptes scabies.

Without being limited to any particular mechanism, administering a chewing gum to a subject may prevent infection by preventing formation of dental plaque and resulting gingivitis. The effect of chewing gum on plaque and oral infection, such as gingivitis and periodontitis, may include activation of the salivary defense mechanism which works to limit bacterial growth through the flow of fluid along the surfaces of the teeth and oral cavity. Flavors, such as peppermint, lemon and herbal flavors may further stimulate salivary secretion to enhance the effect of the salivary defense mechanism. Other flavors such as menthol eucalyptol, thymol, methyl salicylate, licorice, and cinnamic aldehyde may have inherent germ-killing properties. Reducing levels of bacteria in the oral cavity prevents development of gingivitis and periodontal disease in the mouth, but can also prevent infection in other areas of the body, including the blood. For example, chewing gum may decrease the likelihood that bacteria from gingivitis or other periodontal conditions in the oral cavity may cause infection to occur in other organs or systems in the body such as the lungs where pneumonia or other types of infection may result. In fact, pneumonias are among the most common nosocomial or iatrogenic infections.

To this end, a chewing gum may be administered to an individual at risk for infection such as a nosocomial or iatrogenic infection.

The chewing gum of the present invention may include a variety of chewing gum formulations and excipients. The chewing gum may include, for example, a water-insoluble gum base and a water-soluble portion. The insoluble gum base may comprise elastomers, resins, fats and oils, softeners, and inorganic fillers. The gum base may or may not include wax. The insoluble gum base may constitute approximately 5 to about 95 percent, by weight, of the chewing gum. In an embodiment, the gum base may comprise about 10 to about 50 percent of the chewing gum, and, alternatively, about 20 to about 35 percent, by weight of the chewing gum.

In an embodiment, the chewing gum of the present invention may comprise about 20 to about 60 weight percent synthetic elastomer, 0 to about 30 weight percent natural elastomer, about 5 to about 55 weight percent elastomer plasticizer, about 4 to about 35 weight percent filler, about 5 to about 35 weight percent softener, and/or optional minor amounts (about one percent or less) of miscellaneous ingredients such as colorants, antioxidants, etc.

Synthetic elastomers may include, but are not limited to, polyisobutylene with a weight average molecular weight determined by gel permeation chromatography (GPC) of about 10,000 to about 95,000, or from about 50,000 to about 80,000 GPC weight average molecular weight. Synthetic elastomers may also include isobutylene-isoprene copolymer having styrene-butadiene ratios of about 1:3 to about 3:1 polyvinyl acetate having a GPC weight average molecular weight of about 2,000 to about 90,000, polyisoprene, polyethylene, vinyl acetate- vinyl laurate copolymer having vinyl laurate content of about 5 to about 50 percent by weight of the copolymer, and combinations thereof. Styrene-butadiene, for polyvinyl acetate, may range from about 10,000 to 65,000 GPC weight average molecular weight. In an embodiment, a higher molecular weight polyvinyl acetate is used in the chewing gum base. For vinyl acetate-vinyl laurate, the vinyl laurate content may range from about 10 to about 45 percent.

Natural elastomers may include natural rubber such as smoked or liquid latex and guayule, as well as natural gums, such as jelutong, lechi caspi, perillo, sorva, massaranduba balata, massaranduba chocolate, nispero, rosindinha, chicle, gutta hang kang, and combinations thereof. Synthetic elastomers and/or natural elastomer concentrations may vary depending on whether the chewing gum in which the base is used is adhesive or conventional, or a bubble gum or a conventional gum.

Elastomer plasticizers may include, but are not limited to, natural rosin esters, or estergums, such as glycerol esters of partially hydrogenated rosin, glycerol esters polymerized rosin, glycerol esters of partially dimerized rosin, glycerol esters of rosin, pentaerythritol esters of partially hydrogenated rosin, methyl and partially hydrogenated methyl esters of rosin, and pentaerythritol esters of rosin. Synthetic elastomer plasticizers may include terpene resins derived from alpha-pinene, beta-pinene, and/or d-limonene; and any suitable combinations thereof. The elastomer plasticizers may vary depending on the specific application and on the type of elastomer included in the chewing gum.

The chewing gum may also include fillers/texturizers such as magnesium and calcium carbonate; ground limestone; silicate types, such as magnesium and aluminum silicate; clay; alumina; talc; titanium oxide; mono-, di- and tri-calcium phosphate; cellulose polymers, such as wood; and combinations thereof.

The chewing gum may also include softeners or emulsifiers such as tallow, hydrogenated tallow, hydrogenated and partially hydrogenated vegetable oils, cocoa butter, glycerol monostearate, glycerol triacetate, lecithin, mono-, di- and triglycerides, acetylated monoglycerides, fatty acids (e.g. stearic, palmitic, oleic and linoleic acids), and combinations thereof.

The chewing gum may also include colorants and whiteners such as FD&C-type dyes and lakes, fruit and vegetable extracts, titanium dioxide, and combinations thereof.

In addition to a water-insoluble gum base portion, the chewing gum composition may include a water-soluble bulk portion and one or more flavoring agents. The water-soluble portion may include bulk sweeteners, high intensity sweeteners, flavoring agents, softeners, emulsifiers, colors, acidulants, fillers, antioxidants, and other components that provide desired attributes.

The softeners, which are also known as plasticizers and plasticizing agents, may constitute between approximately 0.5 to about 15% by weight of the chewing gum. The softeners may include, but are not limited to, carprenin, glycerin, lecithin, and combinations thereof. Aqueous sweetener solutions, such as solutions containing sorbitol, hydrogenated starch hydrolysates, corn syrup and combinations thereof, may also be used as softeners and binding agents in the chewing gum.

Bulk sweeteners may include both sugar and sugarless components. Bulk sweeteners may constitute about 5 to about 95% by weight of the chewing gum. In an embodiment, the bulk sweetener may constitute about 20 to about 80% by weight, or about 30 to about 60% by weight of the gum.

Sugar sweeteners may include saccharide-containing components commonly known in the chewing gum art, such as sucrose, dextrose, maltose, dextrin, dried invert sugar, fructose, levulose, galactose, corn syrup solids, and the like, either alone or in combination. Additionally or alternatively, sugarless sweetners may be used in the chewing gum, such as bulk polyol sweetners or any other suitable sugarless sweetener or combination thereof. Sugarless sweeteners may include, but are not limited to, sorbitol, mannitol, xylitol, hydrogenated starch hydrolysates, maltitol, lactitol, and the like, alone or in combination.

High intensity artificial sweeteners may also be used in combination with the above including, but not limited to, sucralose, aspartame, aspartame derivatives and conjugates, such as neotame, salts of acesulfame, alitame, saccharin and its salts, cyclamic acid and its salts, glycyrrhizin, dihydrochalcones, thaumatin, monellin, and the like, alone or in combination. In order to provide longer lasting sweetness and flavor perception, it may be desirable to encapsulate or otherwise control the release of at least a portion of the artificial sweetener. Such techniques as wet granulation, wax granulation, spray drying, spray chilling, fluid bed coating, coacervation, and fiber extension or other methods known in the art may be used to achieve the desired release characteristics.

The amount of the artificial sweetener may vary greatly and may depend on such factors as potency of the sweetener, rate of release, desired sweetness of the product, level and type of flavor used and cost considerations. Thus, the level of artificial sweetener may vary from about 0.02 to about 8% by weight of the chewing gum. When carriers used for encapsulation are included, the amount of the encapsulated sweetener may be proportionately higher.

Combinations of sugar and/or sugarless sweeteners may be used in chewing gum. Additionally, the softener may also provide additional sweetness such as with aqueous sugar or alditol solutions.

If a low calorie gum is desired, a low caloric bulking agent may be used. Examples of low caloric bulking agents may include polydextrose, raftilose, raftilin, fructooligosaccharides (NutraFlora), palatinose oligosaccharide, guar gum hydrolysate (Sun Fiber), or indigestible dextrin (Fibersol). However, any other suitable low-calorie bulking agent may be used.

The chewing gum may also include at least one flavoring agent. The flavor may be used in amounts of approximately 0.1 to about 15 weight percent of the gum, and in an embodiment, about 0.2 to about 5%. The flavorant or flavor may include any natural or synthetic oil and/or flavor as is commonly known in the art. Suitable flavorants include, but are not limited to, oils derived from plants and fruits such as citrus oils; fruit essences; fruit juices, fruit concentrates, fruit purees, of, for example, berry, lemon, lime, strawberry, orange, apple and the like; peppermint oil; spearmint oil; other mint oils; clove oil; oil of wintergreen; anise and the like; flavors derived from spices and the like; flavor oils such as menthol eucalyptol, thymol, methyl salicylate, licorice, cinnamic aldehyde and the like; and combinations thereof. The chewing gum may also include herbal flavors such as herbal extracts. Herbal extracts may include eucalyptus, clary sage, marjoram, rosemary, thyme, chamomile, lavender, myrrh or any other suitable polyol sweetener or combinations thereof. Artificial flavoring agents and components may also be used as the flavorant or flavor. Natural and artificial flavoring agents may be combined in any sensorally acceptable fashion as is commonly known in the art. The flavorant or flavor may be encapsulated or non-encapsulated. Encapsulated flavorant may be used to increase or decrease the flavor release rate as is commonly known in the art. In an embodiment, a flavor, such as a peppermint flavor, an herbal flavor, a lemon flavor or combinations thereof, is included in the chewing gum to contribute to reducing infection.

The chewing gum may or may not include an active ingredient or medicament. As used herein, “medicament” may include a pharmacologic or therapeutic agent or component or metabolite thereof that demonstrates pharmacological activity or other direct effect in the diagnosis, cure, mitigation, treatment, or prevention of a condition, or disease. The medicament may include an ingredient that is typically not a component of a chewing gum as described above. For example, a medicament may exclude components such as flavors.

The chewing gum may or may not include an infection-reducing agent or medicament. As used herein, an infection-reducing medicament refers to a medicament that at least contributes to reducing infection. In an embodiment, in the absence of an infection-reducing medicament, at least one flavor, such as a peppermint flavor, an herbal flavor, a lemon flavor or combination thereof, is included in the chewing gum to enhance the effect of the chewing gum to reduce infection. However, it is believed that the chewing gum can be effective in the present invention even in the absence of such active ingredients. In an embodiment, the chewing gum may include active ingredients or medicaments other than an infection-reducing medicament.

The chewing gum center may be manufactured by any suitable method known in the art. Once the chewing gum center is produced, the chewing gum center may or may not be coated or surface-treated by any suitable method known in the art. In an embodiment, the chewing gum may include a coated chewing gum comprising a gum center and a sugar or sugar alcohol coating. The coating may initially be in a liquid state, such as a syrup, which contains the coating ingredients previously described herein in an amount from about 30% to about 85% by weight of the coating, and a solvent, such as water, in an amount from about 15% to about 70% by weight of the coating. In an embodiment, the coating material or syrup is applied or distributed over the gum center one or more times providing additional coating material or syrup II to produce one or more coatings or layers of coating as desired. In an embodiment, the coated chewing gum product may contain about 10% to about 65% coating.

In an embodiment, a soft coating may be formed by adding a powder coating after a liquid coating. The powder coating may include natural carbohydrate gum hydrolysates, maltodextrin, gelatin, cellulose derivatives, starches, modified starches, sugars, sugar alcohols, natural carbohydrate gums and fillers such as talc, calcium carbonate and the like, and combinations thereof.

By way of example, and not limitation, examples of the chewing gum formulations suitable for use in the present invention are set forth in Table 1 below.

TABLE 1

| | Example 1- | Example 2- | Example 3- | |
|---|---|---|---|---|
| | Herbal | Herbal | Peppermint | Example 4- |
| | Flavor | Flavor | Flavor | No Flavor |
| Center | | | | |
| Gum Base | 48.00 | 42.50 | 47.60 | 42.50 |
| Sorbitol | 46.00 | 48.50 | 43.14 | 48.92 |
| Glycerin | 3.20 | 6.00 | 7.44 | 6.00 |
| Triacetin | 0.50 | 0.30 | — | 1.25 |
| Lecithin | — | — | — | 0.45 |
| Herbal Lemon | 1.50 | 1.80 | — | — |
| Mint Flavor | | | | |
| Balm Mint | — | 0.02 | — | — |
| Powder Extract | | | | |
| Peppermint | — | — | 1.22 | — |
| Flavor | | | | |
| Menthol | 0.30 | — | 0.47 | — |
| Acesulfam K | 0.01 | 0.02 | — | 0.02 |
| Encapsulated | 0.25 | — | — | — |
| Acesulfam K | | | | |
| Encapsulated | 0.25 | 0.86 | 0.13 | 0.86 |
| Aspartame | | | | |
| Total Center | 100.00 | 100.00 | 100.00 | 100.00 |
| Coating | | | | |
| Xylitol | 88.99 | 89.05 | 88.27 | 89.65 |
| Gum Acacia | 9.0 | 9.20 | 9.22 | 9.20 |
| Color | 0.81 | 0.82 | 0.83 | 0.82 |
| Peppermint | — | — | 1.00 | — |
| Flavor | | | | |
| Herbal Lemon | 0.87 | 0.60 | — | — |
| Mint Flavor | | | | |
| Menthol | — | — | 0.35 | — |
| Talc | 0.18 | 0.18 | 0.18 | 0.18 |
| Carnauba Wax | 0.15 | 0.15 | 0.15 | 0.15 |
| Total Coating | 100.00 | 100.00 | 100.00 | 100.00 |

The finished product is approximately 67.5% center and 32.5% coating.

The chewing gum may be administered to subjects who have an oral infection. In particular, the methods of the present invention are especially suited for subjects lacking proper oral care. A subject may lack proper oral care if the subject is unable to practice proper procedures for caring for his teeth and gums such as brushing and flossing his teeth. For example, a subject may be unable to physically perform the required motions to properly clean his teeth, such as a patient in a hospital or in any other long-term or short-term care facility. A further example of a subject lacking proper oral care may include one who improperly or ineffectively brushes and flosses his teeth due to inadequate technique or instrumentation, or inaccessible dentition. While the methods of the present invention may be applied to individuals lacking proper oral care, it should also be appreciated that the methods of the present invention may be used to supplement proper oral care.

A therapeutically effective amount or the amount of chewing gum provided or administered to a subject at a particular time may include any amount able to be chewed at one time by the subject. For example, the amount of chewing gum administered to a subject may include about 1 to about 5 grams of chewing gum. In an embodiment, about 2 to about 4 grams is administered. In an embodiment, about 3 grams of chewing gum is administered to a subject. The amount of chewing gum administered to a subject may include any number of pieces of chewing gum necessary to achieve the desired amount.

Chewing gum may be administered to a subject at any suitable frequency and duration. In an embodiment, the chewing gum is administered to a subject about three times per day. In an embodiment, the chewing gum is administered to a subject after ingestion of food or fluids such as after a meal. Administering a chewing gum following a meal may also have the benefit of promoting the removal of remnants of the meal from the oral cavity which would otherwise contribute to the formation of the biofilm precursor to plaque. The gum may be chewed for any suitable period of time such as at least about ten minutes. In an embodiment, the gum is chewed for at least about twenty minutes.

To assess the effect of chewing gum on reducing infection and, in particular, reducing dental plaque accumulation and gingivitis, two double-blinded studies were performed on parallel groups of suitable subjects. The studies used a 21-day, partial mouth gingivitis model described below to accelerate plaque formation and gingivitis development. In one study, a group administered chewing gum was compared to a group not administered chewing gum. In the second study, four parallel groups were evaluated and compared. Each of the four groups was provided either a sugar-free chewing gum with herbal flavoring, a sugar-free chewing gum with no herbal flavoring, a commercial sugar-free chewing gum, or no chewing gum as a control. The general formula for the gums used in the study included about 20% to about 27% gum base; about 50% to about 68% bulk polyol sweeteners such as sorbitol, xylitol, or mannitol; about 0.1% to about 0.4% high-intensity sweeteners such as aspatame, acesulfame K; about 10% to about 23% filler ingredients such as calcium carbonate; and about 0.5% to about 2.9% flavors, including herbal extracts. The herbal-flavored chewing gum is made with food-approved ingredients currently used in sugar-free chewing gums. The herbal mint flavoring was based on common chewing gum flavors with the addition of herbal flavors such as eucalyptus, clary sage, ma roram, rosemary, thyme, chamomile, lavender, and myrrh. The placebo gum was similar in composition to the test gums, but did not contain any herbs or herbal flavor.

The subjects were distributed into equivalent groups of about forty individuals according to three variables: gender, baseline gingival bleeding and inflammation scores of a designated test portion of the mouth. The subjects were also distributed to produce groups with the same potential for forming plaque and developing experimental gingivitis in order to minimize the sample variance within groups.

In an effort to optimize oral health prior to beginning a three-week period of testing, each of the subjects underwent a screening exam and were provided oral hygiene instructions instructing them to perform daily brushing with a regular commercial dentifrice and flossing. Immediately prior to the trial period, each subject underwent prophylaxis including scaling and polishing of the entire dentition. Plaque and gingivitis scores from this pre-trial period were then used as a baseline for longitudinal comparisons and for assignment of subjects to treatment groups for the trial period.

During the three-week trial period, all subjects were asked to abstain from all oral hygiene procedures other than those performed as part of the study and not to use any other unassigned dental products or chewing gum. In addition, the subjects were not permitted to brush the dentition in a portion of the mouth to be tested but were allowed to brush the remainder of the dentition twice daily using the assigned dentifrice. The test quadrant was selected based on the lack of interfering restorations, missing teeth, or other abnormalities. In most cases, the test teeth comprised five teeth from canine to the second molar inclusive.

A removable tooth shield was placed over the test portion to prevent the mechanical action of a toothbrush from reaching the teeth and gums of the test area during tooth brushing. Tooth shields were constructed from vacuum-formed mouth-guard plastic and shaped according to a dental impression taken of the mandibular teeth of each subject. The tooth shield was trimmed to include only the teeth and gingival margin and to eliminate contact with the cervical margin of each tooth, thereby reducing the risk of plaque being disturbed during insertion or removal of the shield. The material was trimmed vertically on the buccal side to a length just short of the vestibule and frenum attachments, and on the lingual side to a length short of the floor of the mouth. The material was further trimmed mesially to the middle of the lateral incisor, and distally behind the second molar.

After brushing with the shield in position, subjects carefully removed the tooth shield to avoid scraping off any deposits, and rinsed the mouth once with tap water. Those subjects assigned a chewing gum were advised to chew two pieces (approximately 3 grams) of the assigned gum three times per day (after breakfast, lunch, and dinner) for twenty minutes so that the effect of subsequent chewing of the gum could be assessed.

At the end of the three-week trial period, clinical assessments were repeated for soft tissue, plaque, and gingivitis as performed during the baseline examination.

The primary results of the study are presented in terms of scores describing plaque accumulation and level of gingivitis using Plaque, Modified Gingival and Gingival Bleeding Indices. The scores for these indices were summed and averaged to provide mean per site scores for each subject at each clinical examination. Data from the gingivitis and plaque indices were grouped and analyzed separately according to the shield-protected teeth and the corresponding brushed teeth. Data for each scoring index was analyzed by analysis of covariance using the baseline (pre-trial) data as the covariate. The covariate (baseline data) was included in the statistics model for increased precision in determining the effect of the test products on the scores. Any variations between treatment groups that existed in the baseline data were compensated for by adjusted means generated by this procedure. Statistical significance of mean data for age and compliance was determined parametrically by analysis of variance for testing of differences between the product groups. Longitudinal (within-treatment) comparisons were also performed for scoring index means using a one-sample t-test on the changes from baseline to the final examination. All comparisons were tested at an overall 0.05 level of significance using two-sided tests.

Plaque and gingivitis was scored on the buccal and lingual surfaces of the five posterior mandibular teeth (i.e. the canine through second molar inclusive) that were protected by the tooth shield, as well as the five contralateral teeth in the opposite mandibular quadrant that are treated by tooth brushing. Adjacent teeth or other suitable teeth were substituted for any unavailable or non-scorable teeth.

Supragingival dental plaque accumulation on the teeth was measured by means of a modification of the Turesky et al. refinement of the Quigley-Hein index (MQHI). MQHI is a numerical index based on plaque area that focuses on the gingival third of the tooth. This weighting of score to differentiate relatively subtle amounts of plaque enables it to reflect the realities of the plaque-gingival inflammation relationship rather than just aesthetic considerations. To conduct the assessment, plaque was first stained with a disclosant and scored according to a five-point interval scale in which the higher value denotes a quantitative increase in plaque. Each tooth was visually divided into six areas for scoring: 1) mesio-facial, 2) mid-facial, 3) disto-facial, 4) mesiolingual, 5) mid-lingual, and 6) disto-lingual. Thus, the maximum score per tooth is 30. If there was no visible plaque, the MQHI score=0. If there were separate flecks of plaque at the cervical margin of the tooth, the MQHI score=1. If there was a thin, continuous band of plaque up to 1 mm wide at the cervical margin, the MQHI score=2. If there was a band of plaque wider than 1 mm but covering less than one-third of the crown, the MQHI score=3. If there was plaque covering at least one-third but less than two-thirds of the crown, the MQHI score=4, and if plaque covered two-thirds or more of the crown, the MQHI score=5. The scores from the six areas of the tooth were summed and divided by 6 to give the mean score for the tooth. Finally, by adding the indices for the teeth and dividing by the number of teeth examined, the mean score for the individual was obtained.

Inflammation of the gingival tissue was measured visually using the Modified Gingival Index (MGI). The gingival margin was defined as the portion located on the enamel or at various levels apical to the cemento-enamel junction. Although the margin should be thin, the buccal and lingual gingiva may present a rounded termination against the tooth, thereby forming the entrance or orifice of the gingival crevice. The MGI included the visible symptoms of gingival inflammation as defined by Lobene. In particular, the marginal and papillary gingival segments of each tooth were defined as clinically healthy having no inflammation if gingiva color was pale pink to pink, the surface after drying is matt with varied degree of stippling, and the gingiva was firm upon palpation with a pocket probe. Accordingly, no inflammation was observed, the MGI score=0. If there was mild inflammation having a slight change in color, little change in texture of any portion of the marginal or papillary gingival unit, but not the entire gingival unit, the MGI score=1. If the mild inflammation involved the entire marginal or papillary gingival unit the MGI score=2. If there was glazing, redness, edema, and/or hypertrophy of the marginal or papillary gingival unit, the MGI score=3 for moderate inflammation. In the presence of marked redness, edema and/or hypertrophy of the marginal or papillary gingival unit, spontaneous bleeding, congestion, or ulceration of the gingival, the MGI score=4 for severe inflammation.

Gingival bleeding was assessed according to the Gingival Bleeding Index (GBI) which indicates the tendency of bleeding of the gingiva upon gentle stroking with a probe along the inner wall of the gingival crevice using a refinement of the GBI by Ainamo and Bay. The principle of the GBI as defined by Saxton and van der Ouderaa is that bleeding is the most significant parameter of gingival inflammation and that the number of elicited bleeding points represents the gingival condition. Severity of bleeding was assessed based upon the ease with which bleeding was elicited by a blunt, periodontal probe. Severity was proportional to the time required to observe bleeding after probing. The periodontal probe was inserted into the gingival crevice and moved around the crevice, gently stretching the sulcular epithelium. Each of the three gingival areas (i.e. mesial, buccal, and lingual) of the test teeth in one quadrant were probed in this manner before recording the number of gingival units which bleed. If no bleeding of the gingival units is observed after 30 seconds, the GBI score=0. If bleeding is observed after 30 seconds, the GBI score=1. If bleeding occurs instantaneously, the GBI score=2. The number of elicited bleeding points are totaled and divided by the units probed (maximum=28) to provide a mean score.

The use of the tooth shield to protect the test teeth from mechanical oral hygiene resulted in significant increases in plaque and gingivitis for each of the treatment groups. Specifically, the baseline MQH scores for each of the groups in the two studies ranged between 2.39 and 2.89 with no significant difference between the groups. After the three-week test period, a significant increase in MQH scores occurred for each group to a range of between 3.27 and 3.59. The baseline MGI scores for each of the groups in the first study ranged between 0.83 and 0.88 with no significant difference between the groups. After the three-week test period, a significant increase in MGI scores occurred for each group to a range of between 1.18 and 1.48. Similarly, in the second study, the baseline MGI scores significantly increased from a range of between 1.21 and 1.28 to a range of between 1.44 and 1.80 after the three-week test period. The baseline GBI scores for each of the groups in the two studies ranged between 0.09 and 0.19 with no significant difference between the groups. After the three-week test period, a significant increase in MGI scores occurred for each group to a range of between 0.19 and 0.71. Thus, this clinical model is a valid and effective method for determining the efficacy of therapeutic dental products in preventing the formation of plaque and gingivitis.

The results of the two studies demonstrate that plaque accumulation and gingivitis are reduced in subjects administered chewing gum. More specifically, as illustrated in Table 2, a statistically significant reduction in plaque occurred in the shielded teeth of subjects administered peppermint and herbal flavored gums in comparison to no gum control. Additionally, there was a statistically significant decrease of 7.3% in the Plaque Index scores for the peppermint gum and an 8.9% decrease in the Plaque Index scores for the herbal lemon flavor gum compared to the group not administered chewing gum. Gum with no flavor was not statistically significant in comparison to no gum.

The results of the study also showed that a decrease in gingivitis occurred in subjects administered chewing gum as indicated by a statistically significant decrease in the Gingival Index scores and in the Bleeding Index scores of the subjects in the first study administered the herbal lemon flavor gum (−18% and −26%, respectively) compared to the group not administered chewing gum. A statistically significant decrease also occurred in the Gingival Index scores of the subjects in the second study administered the peppermint gum (—13%), herbal lemon flavor gum (—20%) and unflavored gum (—15%) compared to the group not administered chewing gum. In addition a decrease in the Bleeding Index scores occurred in subjects administered the peppermint gum (—25%), herbal lemon flavor gum (—32%) and unflavored gum (−4%) compared to the group not administered chewing gum. Although the decrease in the Bleeding Index scores was not statistically significant, the combined decrease in gingival index scores and bleeding index scores indicates a significant reduction of gingivitis in subjects administered chewing gum.

The decrease in the Plaque Index scores as well as the Gingival and Bleeding Index scores for unflavored gum further indicates that just chewing non-flavored gum can reduce gingivitis and that the flavor in the gums may have an added effect of decreasing gingivitis and the plaque that causes gingivitis.

TABLE 2
SUMMARY OF FINAL PLAQUE (MQH), GINGIVITIS (MGI), AND
GINGIVAL BLEEDING (GBI) SCORES FOR SHIELDED TEETH

| | | Plaque | | Gingivitis | | | |
|---|---|---|---|---|---|---|---|
| | | Final | Reduction | Final | Reduction | Final | Reduction |
| Test Group | | MQH^{§} | (Significance) | MGI^{§} | (Significance) | GBI^{§} | (Significance) |
| First | Example 1- | 3.43 | 5% | 1.46 | 18% | 0.53 | 26% |
| Study | Herbal Gum | | (p = 0.03) | | (p = 0.001) | | (p = 0.04) |
| | No-Gum | 3.60 | — | 1.78 | — | 0.72 | — |
| Second | Example 2- | 3.27 | 9% | 1.18 | 20% | 0.19 | 32% |
| Study | Herbal Lemon | | (p = 0.007) | | (p = 0.003) | | (p = 0.06) |
| | Example 3- | 3.32 | 8% | 1.29 | 13% | 0.21 | 25% |
| | Peppermint | | (p = 0.01) | | (p = 0.03) | | (none) |
| | Example 4- | 3.37 | 6% | 1.26 | 15% | 0.27 | 4% |
| | Unflavored | | (none) | | (p = 0.02) | | (none) |
| | No-Gum | 3.59 | — | 1.48 | — | 0.28 | — |

^{§}Adjusted mean score after 3 weeks of treatment, n˜35 per group

As indicated in Table 3 below, the further positive effect of the chewing gums on unshielded teeth maintained with regular brushing and flossing after only three weeks further indicates that the chewing gums may provide some additional oral hygiene benefits beyond regular oral care.

TABLE 3
SUMMARY OF FINAL PLAQUE (MQH), GINGIVITIS (MGI), AND
GINGIVAL BLEEDING (GBI) SCORES FOR NON-SHIELDED TEETH

| | | Plaque | | Gingivitis | | | |
|---|---|---|---|---|---|---|---|
| | | Final | Reduction | Final | Reduction | Final | Recudtion |
| Test Group | | MQH^{§} | (Significance) | MGI^{§} | (Significance) | GBI^{§} | (Significance) |
| First | Example 1- | 2.71 | 0% | 1.31 | 10% | 0.24 | 8% |
| Study | Herbal Gum | | (p = 0.78) | | (p = 0.06) | | (p = 0.66) |
| | No-Gum | 2.69 | — | 1.45 | — | 0.26 | — |
| Second | Example 2- | 2.52 | 10% | 0.97 | 17% | 0.07 | 42% |
| Study | Herbal Lemon | | (p = 0.006) | | (p = 0.01) | | (p = 0.07) |
| | Example 3- | 2.68 | 4% | 1.06 | 9% | 0.09 | 25% |
| | Peppermint | | (none) | | (p = 0.03) | | (none) |
| | Example 4- | 2.60 | 7% | 1.12 | 4% | 0.11 | 8% |
| | Unflavored | | (none) | | (none) | | (none) |
| | No-Gum | 2.79 | — | 1.17 | — | 0.12 | — |

^{§}Adjusted mean score after 3 weeks of treatment, n˜35 per group

The overall safety data collected during this three-week clinical trial also demonstrated that there is little or no risk or significant side effects associated with regular use of the chewing gums described herein.

Therefore, the results of this human clinical investigation demonstrate that use of a chewing gum as the sole method or as a supplemental method of addressing oral hygiene for three weeks provides a safe and effective way to significantly reduce plaque and gingivitis compared to a no-gum control.

It should be understood that various changes and modifications to the embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method of reducing the risk of acquiring a systemic infection comprising:
providing a therapeutically effective amount of a chewing gum without a medicament to a subject having an oral infection.

2. The method of claim 1, wherein the systemic infection is a nosocomial or iatrogenic infection.

3. The method of claim 1, wherein the chewing gum is provided to a patient in a health care facility.

4. The method of claim 1, wherein the chewing gum is provided to a subject lacking proper oral care.

5. The method of claim 1, wherein the chewing gum is provided to a subject as a supplement to oral care.

6. The method of claim 1, wherein the chewing gum includes a flavor selected from the group consisting of an herbal flavor, a lemon flavor, a peppermint flavor, a citrus flavor, a fruit flavor, a spearmint flavor, a mint flavor, a clove flavor, a wintergreen flavor, an anise flavor, a fruit flavor, a spice flavor and combinations thereof.

7. The method of claim 6, wherein the flavor is present in an amount of about 0.2% to about 5% by weight of the chewing gum.

8. The method of claim 6, wherein the herbal flavor includes an herbal extract selected from the group consisting of eucalyptus, clary sage, marjoram, rosemary, thyme, chamomile, lavender, myrrh and combinations thereof.

9. The method of claim 1, wherein the chewing gum is provided to the subject after a meal.

10. A method of reducing the risk of acquiring a second systemic infection comprising:
providing a therapeutically effective amount of a chewing gum without an infection-reducing medicament to a subject having an oral infection.

11. The method of claim 10, wherein the oral infection is gingivitis.

12. The method of claim 10, wherein the systemic infection is a nosocomial or iatrogenic infection.

13. The method of claim 10, wherein the chewing gum is provided to a subject lacking proper oral care.

14. The method of claim 10, wherein the chewing gum is provided to a patient in a health care facility.

15. The method of claim 10, wherein the chewing gum is provided to a subject as a supplement to oral care.

16. The method of claim 10, wherein the chewing gum includes a flavor selected from the group consisting of an herbal flavor, a lemon flavor, a peppermint flavor, a citrus flavor, a fruit flavor, a spearmint flavor, a mint flavor, a clove flavor, a wintergreen flavor, an anise flavor, a fruit flavor, a spice flavor and combinations thereof.

17. A method of reducing the risk of acquiring a secondary infection comprising:
providing a therapeutically effective amount of a chewing gum without an infection-reducing medicament to a subject having a compromised immune system.

18. The method of claim 17, wherein the secondary infection is a nosocomial or iatrogenic infection.

19. A method of reducing the risk of acquiring a nosocomial or iatrogenic infection comprising:
providing a therapeutically effective amount of a chewing gum without a medicament to a subject having a compromised immune system.
